(19) United States
(12) Patent Application Publication
(10) Pub. No.: US 2004/0265832 A1
(43) Pub. Date: Dec. 30, 2004

(54) Model and modeling for predicting a hepatitis B patient to response to interferon treatment

(76) Inventors: Lawrence Shih Hsin Wu, Wugu Shiang (TW); Lichih Huang, Wugu Shiang (TW); Cherry Guan Ju Lin, Wugu Shiang (TW); Julia Kuei Ting Yu, Wugu Shiang (TW); Ding-Shinn Chen, Taipei (TW); Pei-Jer Chen, Taipei (TW); Ming-Yang Lai, Taipei (TW); Shiou-Hwei Yeh, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/601,589

(22) Filed: Jun. 24, 2003

(51) Int. Cl. C12Q 1/70; C12P 21/04; C12Q 1/68; C07H 21/04

(52) U.S. Cl. 435/6; 435/5; 536/23.72

(57) ABSTRACT

By genotyping analysis in combination with Monte-Carlo estimation, a model for predicting an hepatitis B patient to response to interferon treatment is constructed. With this model, by selecting STR markers combination, the hepatitis B patients are divided into three groups comprising high response rate, ambiguous, and low response rate, respectively, and hence, predictions of treatment response for HBV patients, especially for interferon therapy, are obtained.

### FIELD OF THE INVENTION

The present invention relates generally to a method for diagnosing a hepatitis B patient treatment with interferon as having a predisposition to response, or non-response, and a kit therefore. Specifically, the present invention relates to a method that comprises demonstrating in the hepatitis B patient the present or absent of an unusual variant combination form of several STR markers, the unusual variant form being associated with an increased or decreased response rate for hepatitis B patient treated with interferon.

### BACKGROUND OF THE INVENTION

Despite the existence of vaccines, chronic hepatitis B virus (HBV) infection remains a major health problem worldwide. Interferon therapy successfully controls infection in only a small percentage of chronically infected individuals, see Delaney W E 4th, Locarnini S, Shaw T (2001) Resistance of hepatitis B virus to antiviral drugs: current aspects and directions for future investigation, Antivir Chem Chemother 12(1):1-35. Treatment with interferon-alpha leads to cessation of viral replication in 30-40% of patients with chronic hepatitis B, see Heintges T, Petry W, Kaldewey M, Erhardt A, Wend U C, Gerlich W H, Niederau C, Haussinger D (2001) Combination therapy of active HBsAg vaccination and interferon-alpha in interferon-alpha nonresponders with chronic hepatitis B, Dig Dis Sci 46(4):901-906. However, even in patients selected as suitable candidates, the 6 to 12 months of therapy is costly and the numerous side effects can be debilitating, see Liaw Y F (2002) therapy of chronic hepatitis B: current challenges and opportunities, J Viral Hepat 9(6):393-399, Wai C T, Lok A S (2002) Treatment of hepatitis B, J Gastroenterol 37(10):771-778, and Feld J, Locarnini S (2002) Antiviral therapy for hepatitis B virus infections: new targets and technical challenges. J Clin Virol 25(3)267-283. These liabilities have stimulated to search for predictors of response as well as nonresponse to treatment. Candidate predictors now include factors such as viral genotypes, ALT level, serum HBV DNA, female gender, fibrosis on liver biopsy, and serum Fibronectin level, see Kao J H (2002) Hepatitis B viral genotypes: clinical relevance and molecular characteristics, J Gastroenterol hepatol 17(6):643-650, Sakai T. Shiraki K, Inoue H, Okano H, Deguchi M, Sugimoto K, Ohmori S, Murata k, NakanoT (2002) Efficacy of long-term interferon therapy in chronic hepatitis B patients with HBV genotype C, Int J Mol Med 10(2):201-204, Kao J H, Wu N H, Chen P J, Lai M Y, Chen D S (2000) Hepatitis B genotypes and the response to interferon therapy, J Hepatol 3396):998-1002, Neudorf-Grauss R, Bujanover Y, Dinari G, Broide E, Neveh Y, Zahavi I, Reif S (2000) Chronic hepatitis B virus in children in Israel: clinical and epidemiological characteristics and response to interferon therapy, Isr Med Assoc J 2(2):164-168, and Helvaci M, Ozkaya B, Ozbal E, Ozinel S, Yaprak I (1999) Efficacy of interferon therapy on serum fibronection levels in children with chronic hepatitis B infection, Pediatr Int 41(3):270-273.

In focusing on the host genetic background, the role of DNA polymorphism, including STRP and SNP, in associated to disease and treatment response has become increasingly supported in a variety of illnesses. Hence, looking into such a topic may lead to important predictions of treatment response for HBV patients, especially for interferon therapy given the many displeasing side-effects associated with this medical regimen.

### SUMMARY OF THE INVENTION

An object of the present invention is to provide a method of modeling from STR markers for predicting an hepatitis B patient to response to interferon treatment.

Another object of the present invention is to provide a model for predicting an hepatitis B patient to response to interferon treatment by analyzing the STR markers from the hepatitis B patient.

In a modeling method, according to the present invention, genotyping analysis in combination with Monte-Carlo estimation is used to generate a discrimination equation by logistic regression.

The constructed model by the inventive method divides the hepatitis B patients into three groups that are high response rate, ambiguous, and low response rate, respectively, and hence, predictions of treatment response for HBV patients, especially for interferon therapy are obtained.

### BRIEF DESCRIPTION OF THE DRAWINGS

These and other objects, features and advantages of the present invention will become apparent to those skilled in the art upon consideration of the following description of the preferred embodiments of the present invention taken in conjunction with the accompanying drawings, in which:

FIG. 1 shows a flow for modeling for predicting an hepatitis B patient to response to interferon treatment;

FIG. 2 is a graphic representation showing the allelic association on chromosome 1, 2, 3, 4, 5, 6, 7, 8, 9, and 17, in which X-axis is STR markers genetic location on each chromosome from p-terminus to q-terminus, and Y-axis is natural logarithm of Monte-Carlo estimation p value;

FIG. 3 shows the summary information of 14 markers associated with the sustained response of interferon treatment;

FIG. 4 shows the analysis of maximum likelihood estimates in logistic regression on markers D1S2890, D5S406, and D6S1581;

FIG. 5 is the predictor model generated by combined markers D1S2890, D5S406, and D6S1581;

FIG. 6 shows the logistic regression for genotype for the three markers combination;

FIG. 7 is the analysis of maximum likelihood estimates in logistic regression on markers D3S1289, D7S515, D9S288, and D17S785;

FIG. 8 is the predictor model generated by combined markers D3S1289, D7S515, D9S288, and D17S785;

FIG. 9 shows the logistic regression for genotype for the four markers combination;

FIG. 10 is the predictor model generated by combined markers D2S319, D3S1289, D4S391, D7S515, and D17S 785; and

FIG. 11 shows the values of the predictor of FIG. 10.

### DETAILED DESCRIPTION OF THE INVENTION

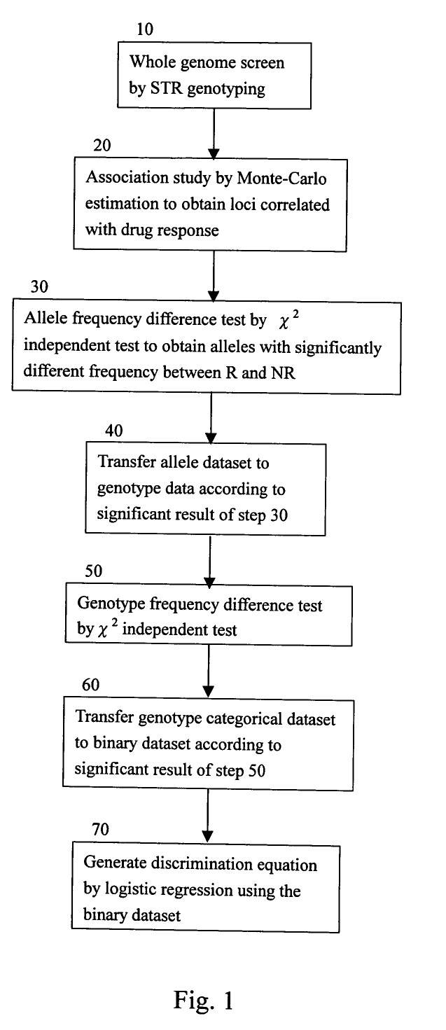

According to the present invention, a model for predicting an hepatitis B patient to response effectively to interferon treatment can be constructed by genotyping analysis in combination with Monte-Carlo estimation, for example, following the procedure shown in FIG. 1. To modeling the predictor, whole genome screen by STR genotyping is performed in step 10 first for collected samples. In step 20, association study is made by Monte-Carlo estimation to obtain loci correlated with drug response. Step 30 is performed for allele frequency difference test by _{X}² independent test to obtain alleles with significantly different frequency between response and non-response to treatment. Then the allele dataset is transferred to genotype data according to significant result in step 40, followed by step 50 for genotype frequency difference test by _{X}² independent test. The genotype categorical dataset is further transferred to binary dataset according to significant result in step 60. Finally, the binary dataset is used to generate discrimination equation by logistic regression.

Examples are provided below to show the inventive method more detailed.

Clinical Sample Collection

It is retrospectively enrolled 104 Chinese Han Patients with chronic hepatitis B from outpatient clinics. All patient blood samples are HBsAg(+) and HBeAg (+) and with an elevated ALT of at least 2 folds higher than the upper limits of normal for six months. Informed consent is obtained in writing from each patient. Patients are excluded from receiving interferon therapy if they had any of the following criteria: neutrophil count <1,500 cells/mm³, Hgb<12 g/dL in women or 13 g/dL in men, or platlet count <90,000 cells/mm³, history of poorly controlled thyroid disease, and serum creatinine level >1.5 times the upper limit of normal at screening. Approximately 30 patients receive liver biopsy before treatment to document active hepatitis or to exclude severe cirrhosis. Eligible patients receive interferon-alpha (2a or 2b) at a dosage of 5-10 MU 3 rimes per week for 4-6 months, and are subsequently followed for treatment response via clinical, biochemical, and serologic markers for more than one year. The definition of sustained responders to IFN treatment for chronic hepatitis B disease includes patients with HBeAg(+) to HBeAg(−) conversion after treatment for at least 1 year after follow-up period. Patients with concurrent hepatitis C or D infection are excluded from the study. The study protocol conforms to the ethical guidelines of the 1975 Declaration of Helsinki as reflected by approval from lour institutional review committee.

Genome-wide Genotyping Analysis

Amplification of STR Marker Fragments from Genomic DNA

Genotyping is performed using the ABI PRISM Linkage Mapping Sets MD-10 (400 markers). These markers are arranged in MD-10 sets to provide coverage of human genome at 10 cM average resolution. Each marker set includes a fluorescence labeled forward primer and a tailing reverse primer. Reverse primer tailing chemistry, by placing the sequence GTTTCTT on the 5′ end of reverse primers, is used to promote the non-template directed nucleotide addition during amplification, which results in consistent allele calls and more precise data output. The PCR reaction containing 9.0 ul True Allele PCR Premix (including dNTPs, buffer, MgCl₂, and Tag DNA polymerase), 3.8 ul sterile deionized water, 1.0 ul primer pair (5 uM each primer), 1.2 ul genomic DNA (50 ng) is prepared on 96-well microtiter plate. Amplification is carried on 9700 PCR machines of ABI with the following thermal reactions: one cycle at 95° C. for 12 minutes, 10 cycles of melting at 94° C. for 15 seconds, annealing at 55° C. for 15 seconds, extending at 72° C. for 30 seconds, 20 cycles of melting at 89° C. for 15 seconds, annealing at 55° C. for 15 seconds, extending at 72° C. for 30 seconds, and one cycle of final extension at 72° C. for 10 minutes.

STR Polymorphism Detection and Analysis

After PCR, pooling the reaction products for a panel of markers at a 1:1:2 ratio (FAM:VIC:NED). Mix 0.5 ul of pooled PCR product with 9 ul of the formamide:size standard mixture, which is prepared by mixing 50 ul of GeneScan-500 LIZ Size Standard with 900 ul of Hi-Di formamide. DNA dispensing, and pooling of PCR products, is performed with separate pipetting robots, ensuring a fast and almost error-free liquid handing process. PCR pools are separated on ABI 3700 DNA Analyzers. The use of GeneScan 500 LIZ as the internal size standard assists polymorphic fragment length calling and allows more accurate allele calling and unambiguous comparison of data across experimental conditions. Genotypes are scored using Genescan and Genotyper (ABI) software.

Genotypes are checked independently by three individuals, without prior knowledge of phenotype.

Statistical Analysis

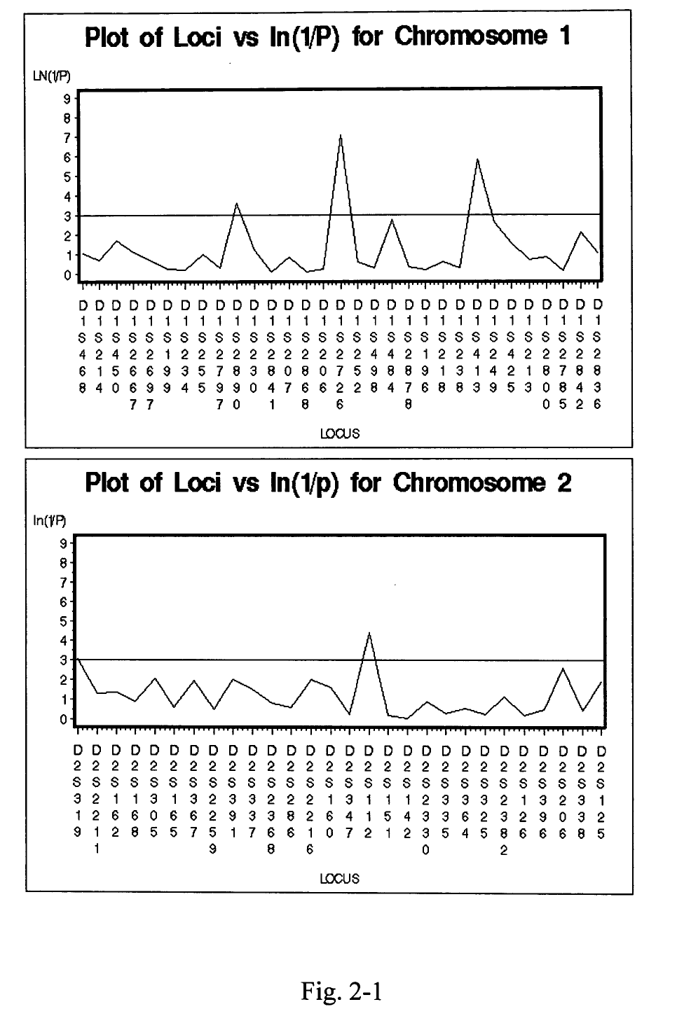

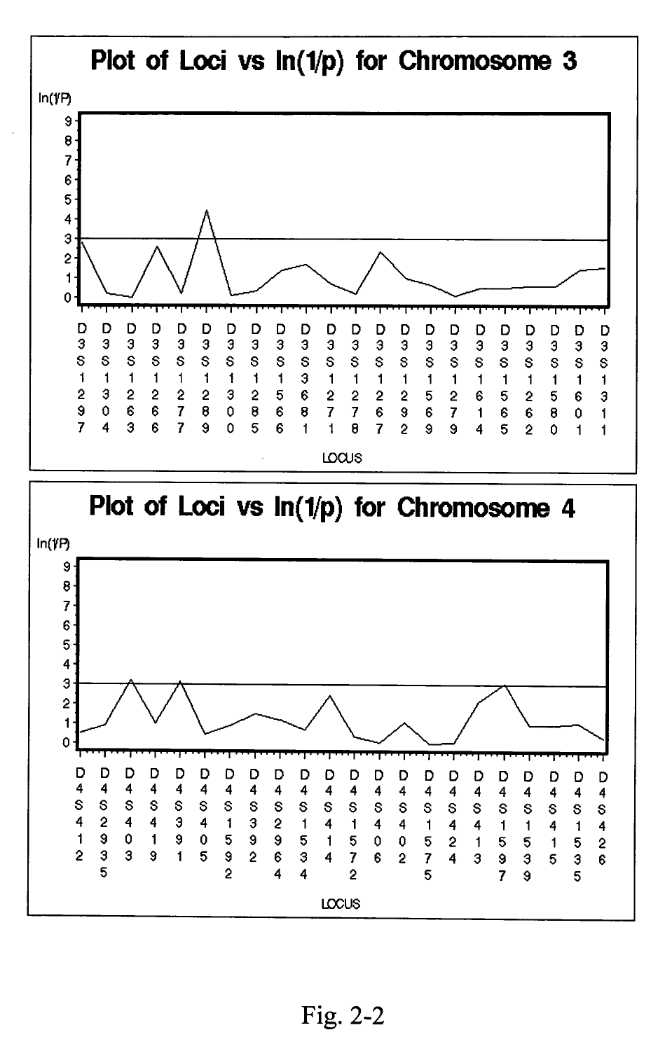

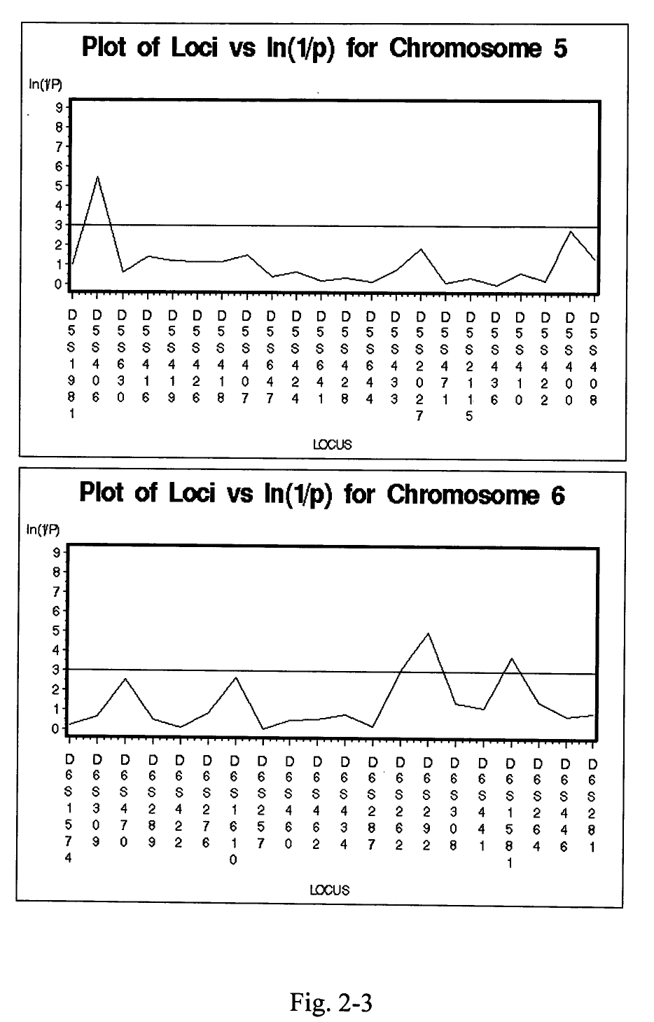

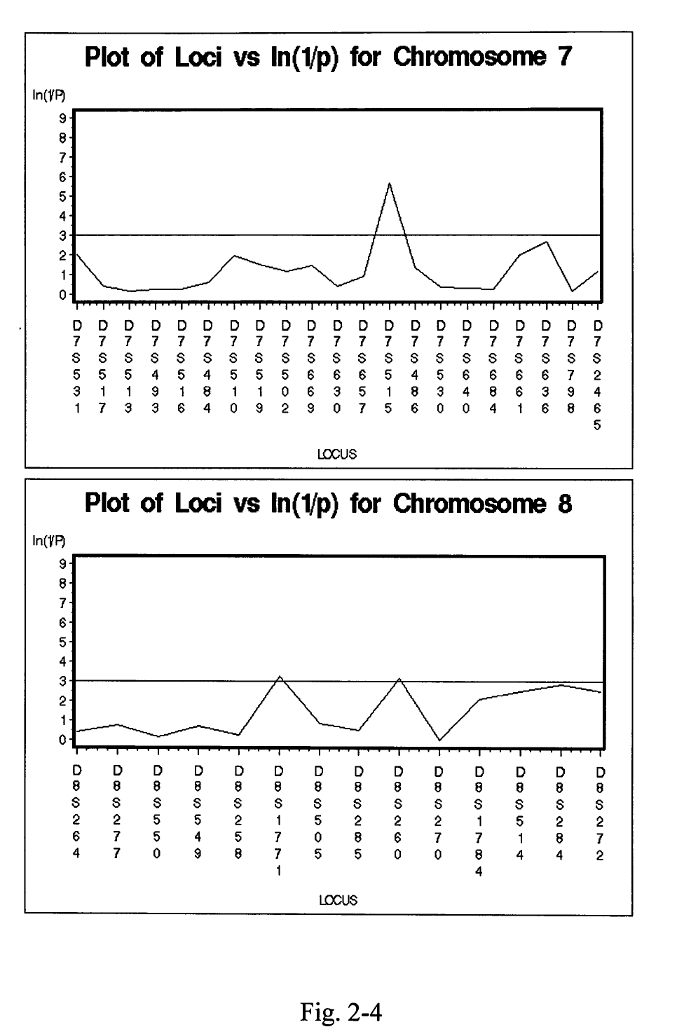

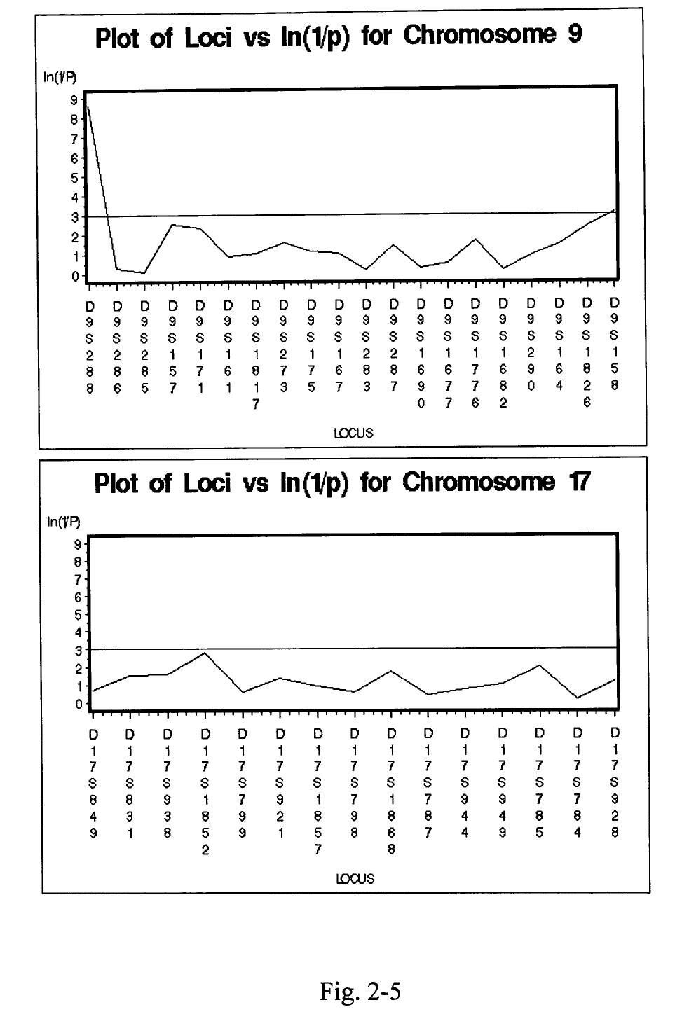

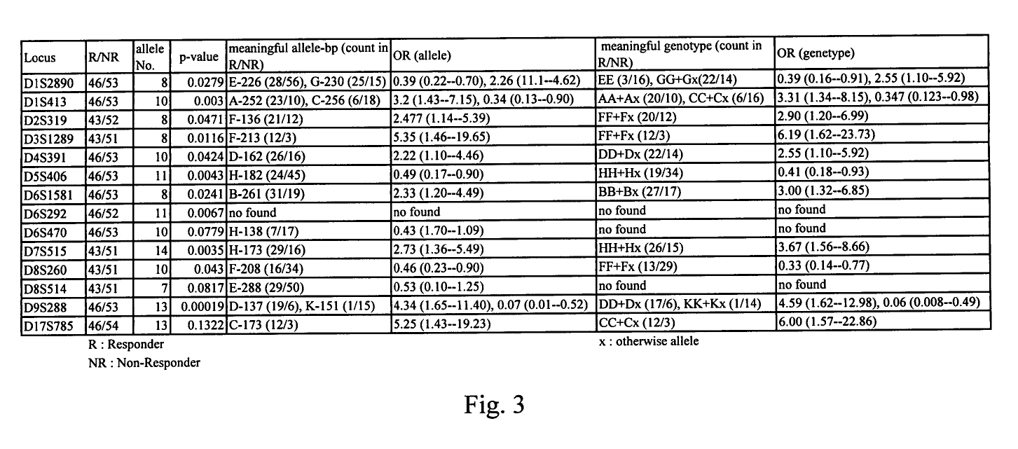

Associations with the sustain response of interferon treatment are sought by Monte-Carlo estimation (SAS10.0, SAS Inc.). FIG. 2 is a graphic representation showing the allelic association on chromosome 1, 2, 3, 4, 5, 6, 7, 8, 9, and 17. In this chart, X-axis is STR markers genetic location on each chromosome from p-terminus to q-terminus, and Y-axis is natural logarithm of Monte-Carlo estimation p value, and of which Y=3 is almost equal to p=0.05. Alleles on significant markers are tested for linkage disequilibrium by analysis of contingency table. The significant alleles are tested for risk factor by odd ratio. Genotype contingency tables are constructed according to specific allele with significant p-value and odd ratio. The meaningful genotype are generated by testing with _{X}² and significant odd ratio to genotype contingency table. After collected all significant markers genotype information and transformed the dataset into binary category, the meaningful genotype and the others genotype are represented by “1” and “0”, respectively. FIG. 3 shows summary information of 14 markers associated with the sustained response of interferon treatment. The information includes the amount of subjects, p-value of Monte-Carlo test, the meaningful allele count, the meaningful genotype count, and odd ratio, etc.

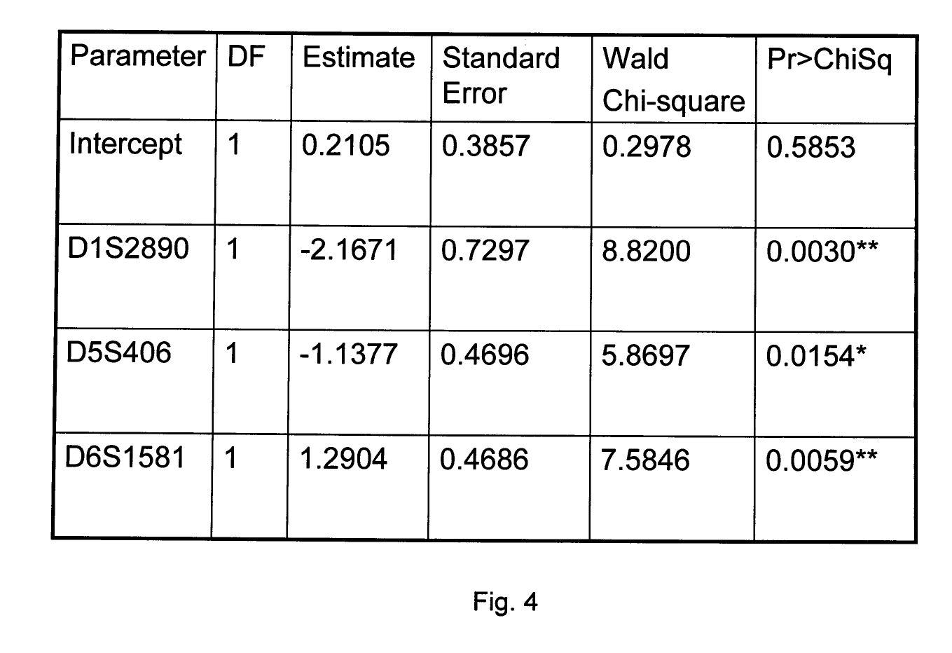

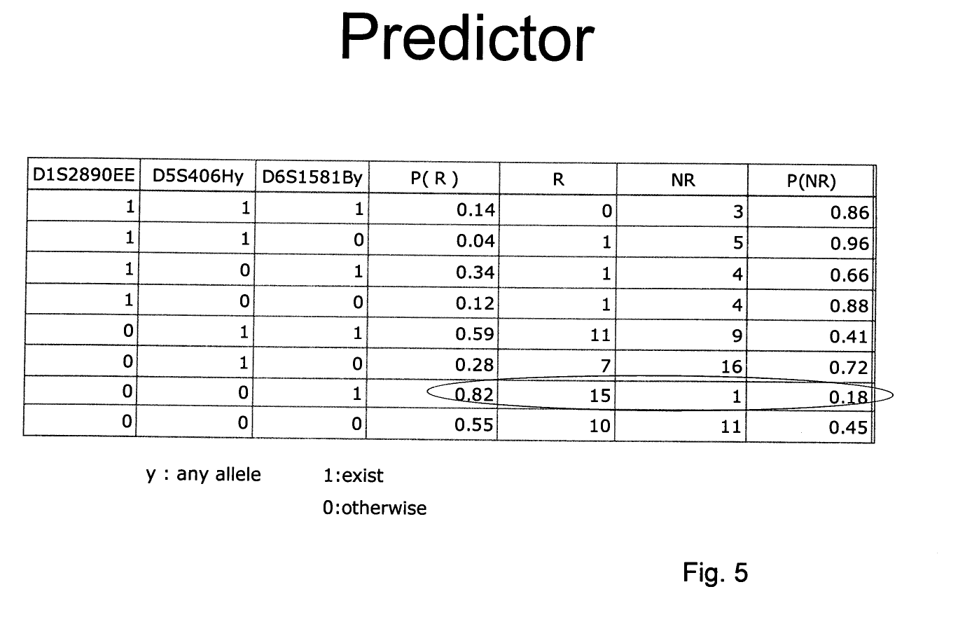

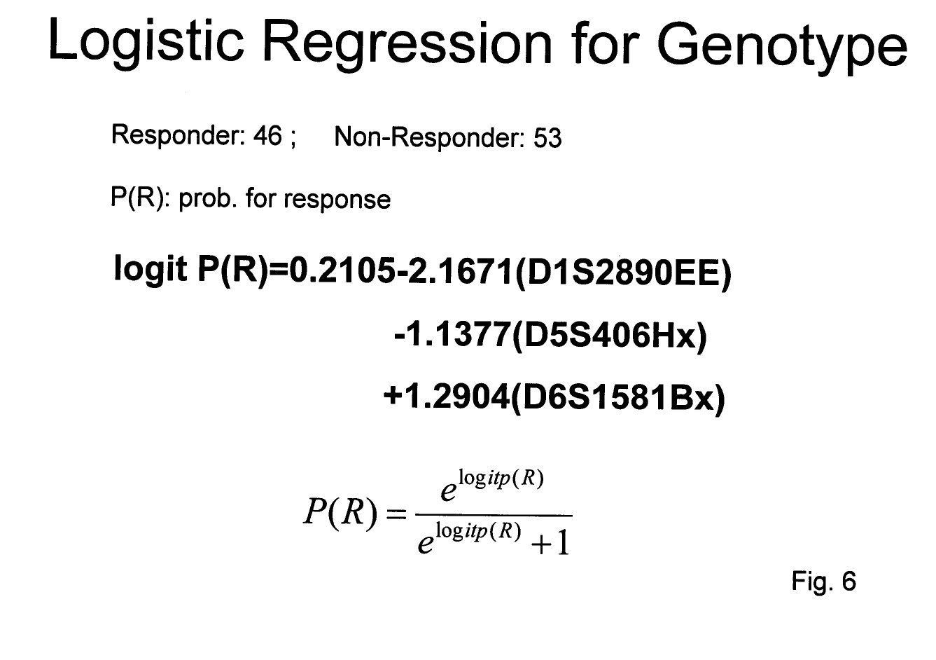

Predictors using the transformed binary dataset from the previous step are performed by logistic regression on variant STR markers combination. FIG. 4 shows the analysis of maximum likelihood estimates in logistic regression on markers D1S2890, D5S406, and D6S1581. FIG. 5 is the predictor model generated by combined markers D1S2890, D5S406, and D6S1581. FIG. 6 shows the logistic regression for genotype in the embodiment of FIGS. 4 and 5. According to these three STR markers combination, 99 subjects (46 responder and 53 non-responder) can be divided into nine groups by

logit P(R)=0.2105−2.1671 (D1S2890EE)−1.1377 (D5S406Hy)+1.2904 (D6S1581By),

where “y” represents any allele. The group with 001 character has the highest response rate.

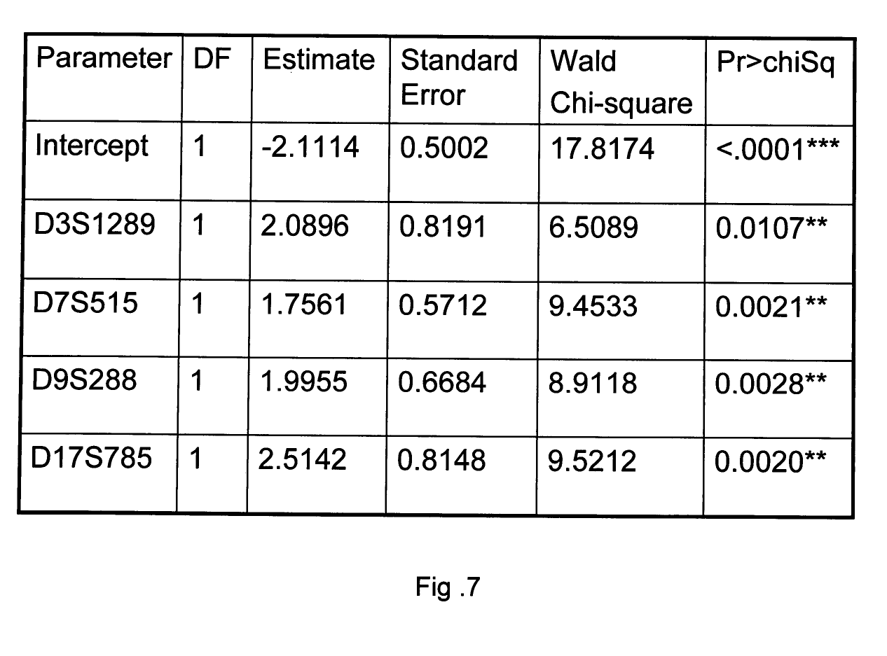

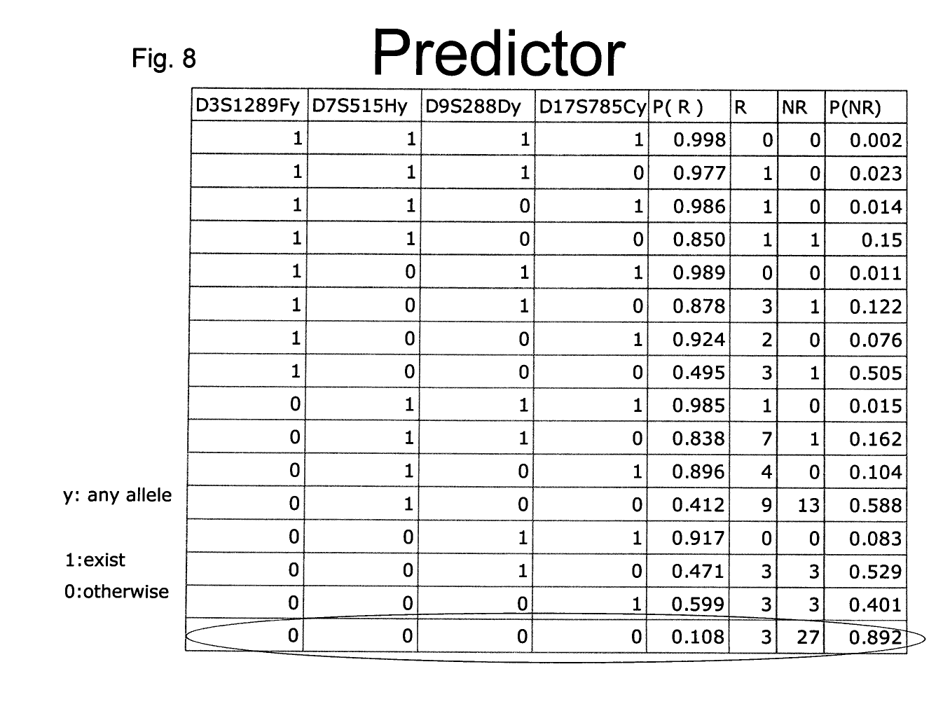

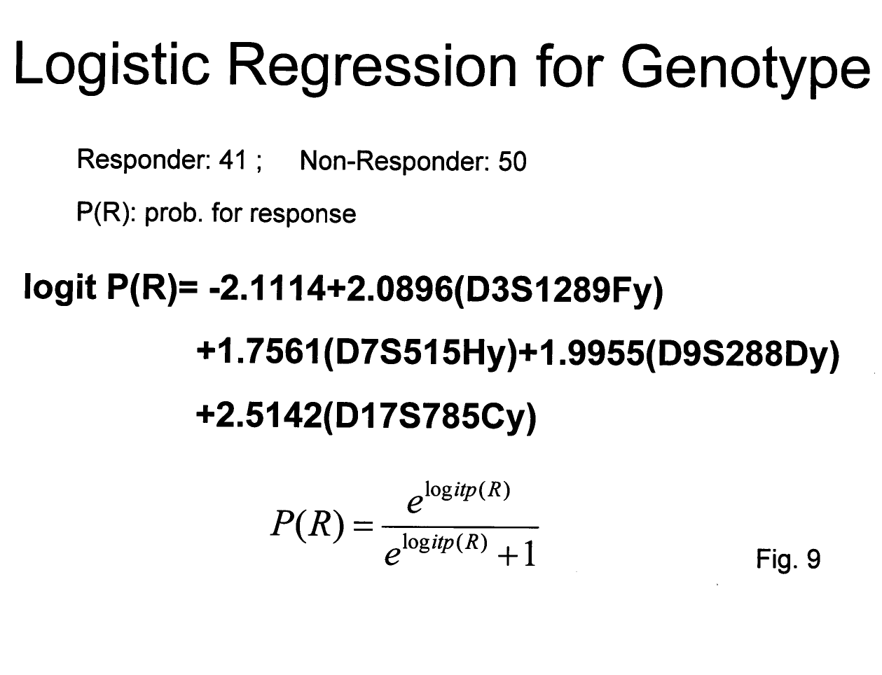

A situation for another combination is shown by FIGS. 7-9. FIG. 7 is the analysis of maximum likelihood estimates in logistic regression on markers D3S1289, D7S515, D9S288, and D17S785. FIG. 8 is the predictor model generated by combined markers D3S1289, D7S515, D9S288, and D17S785. FIG. 9 shows the logistic regression for genotype for this embodiment. According to these four STR markers combination, the patients can be divided into sixteen groups by

logit P(R)=−2.1114+2.0896 (D3S1289Fy)+1.7561 (D7S515Hy)+1.9955 (D9S288Dy)+2.5142 (D17S785Cy),

where “y” represents any allele. The group with 0000 characters has the highest non-response rate.

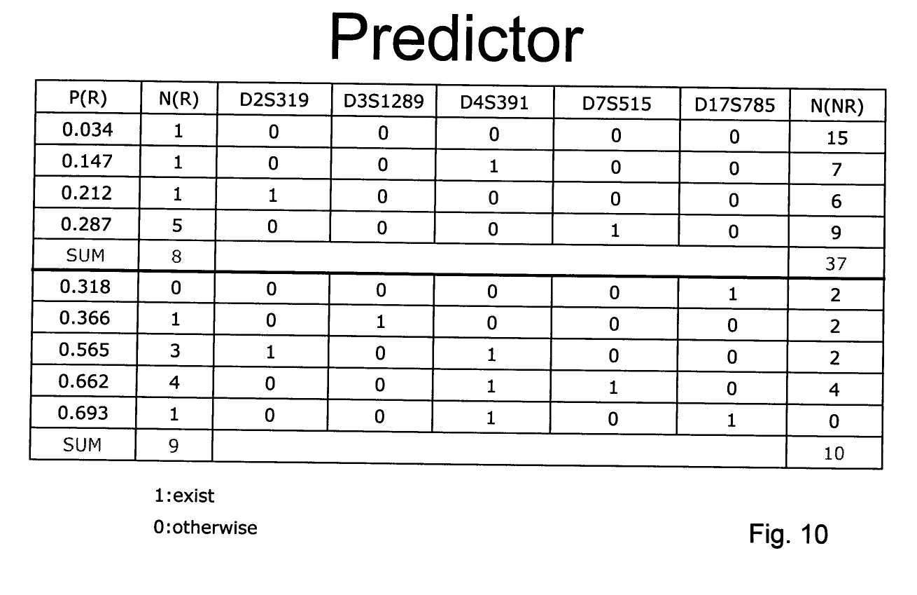

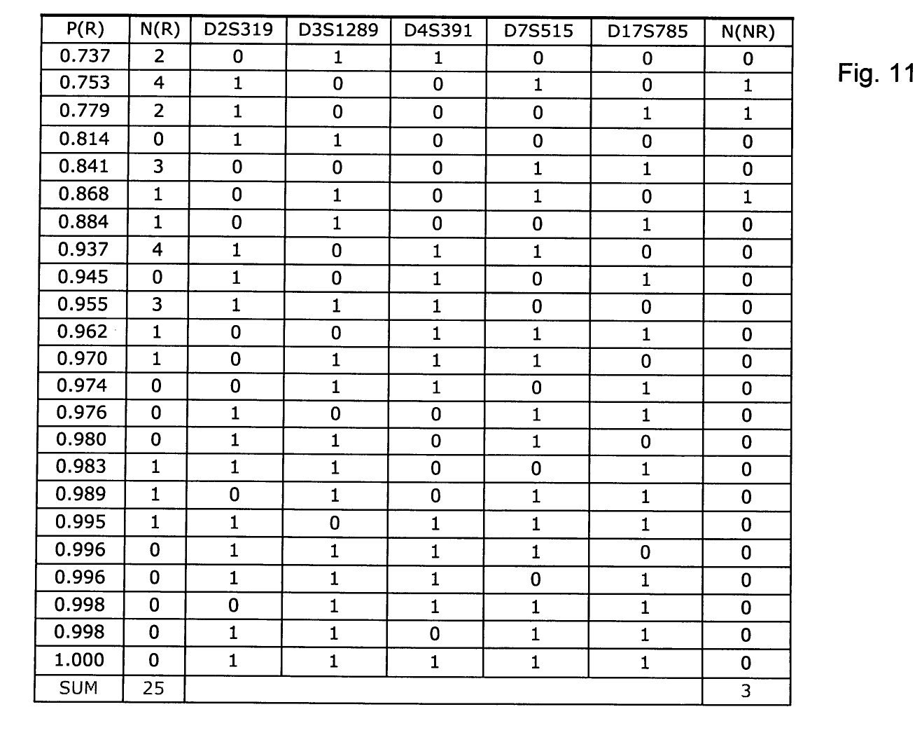

FIG. 10 is the predictor model generated by combined markers D2S319, D3S1289, D4S391, D7S515, and D17S 785. The values of the predictor are shown in FIG. 11. According to this five STR markers combination, the patients can be divided into three groups by

P(R)<0.3,

0.3<P(R)<0.7, and

P(R)>0.7,

following the equation

logit P(R)=−3.3395+2.0239 (D2S319Fy)+2.7922 (D3S1289Fy)+1.5791 (D4S391Dy)+2.4306 (D7S515Hy)+2.5744(D17S785Cy),

where “y” represents any allele.

Results

Genotyping is performed on chromosome 1, 2, 3, 4, 5, 6, 7, 8, 9, and 17 by 215 STR markers (average 10 cM interval). 14 of 215 STR markers' allele frequency or/and genotype frequency are associated with the sustain response of interferon treatment. After all analysis, it is found three STR markers combination available for predicting response rate, and another four STR markers combination available for predicting non-response rate. A five STR markers combination generated by logistic regression without pro-selection, can divide patients into three groups that are high response rate, ambiguous, and low response rate, respectively.

While the present invention has been described in conjunction with preferred embodiments thereof, it is evident that many alternatives, modifications and variations will be apparent to those skilled in the art. Accordingly, it is intended to embrace all such alternatives, modifications and variations that fall within the spirit and scope thereof as set forth in the appended claims.

What is claimed is:

1. A modeling method for predicting an hepatitis B patient to response to interferon treatment, comprising the steps of:
STR genotyping;
associating significant STR markers with response of interferon treatment by Monte-Carlo estimation;
testing alleles on significant STR markers;
transferring significant alleles to genotype information; and
generating an equation based on said genotype information.

2. A method according to claim 1, wherein said STR genotyping comprises the steps of:
amplifying STR marker fragments from genomic DNA; and
detecting and analyzing STR polymorphism.

3. A method according to claim 1, wherein said associating significant STR markers with response of interferon treatment comprises obtaining loci correlated with drug response.

4. A method according to claim 1, wherein said testing alleles comprises analyzing a contingency table.

5. A method according to claim 1, wherein said transferring significant alleles to genotype information comprises constructing a genotype contingency table.

6. A method according to claim 1, wherein said generating an equation comprises transferring said genotype information to a binary dataset.

7. A method according to claim 6, wherein said generating an equation is practiced by a logistic regression.

8. A method according to claim 1, wherein said testing alleles comprises an allele frequency difference test.

9. A method according to claim 1, wherein said transferring significant alleles to genotype information comprises a genotype frequency difference test.

10. A method according to claim 1, further comprising selecting a plurality of STR markers to form a combination for said generating an equation.

11. A method according to claim 10, further comprising selecting a second plurality of STR markers to form a second combination for estimating an error rate for said first combination.

12. A method according to claim 10, wherein said plurality of STR markers includes 5 STR markers.

13. A model for predicting an hepatitis B patient to response to interferon treatment, comprising:
a combination composed of a plurality of STR markers selected from a STR marker set; and
an equation derived from said combination by Monte-Carlo estimation for indicating said hepatitis B patient as high response rate, ambiguous, or low response rate.

14. A model according to claim 13, wherein said combination includes 5 STR markers.
